(19) United States
(12) Patent Application Publication
(10) Pub. No.: US 2023/0225668 A1
(43) Pub. Date: Jul. 20, 2023

(54) SYSTEMS, METHODS, AND MEDIA FOR PREDICTING A CONVERSION TIME OF MILD COGNITIVE IMPAIRMENT TO ALZHEIMER'S DISEASE IN PATIENTS

(71) Applicant: UNIVERSITY OF SOUTH FLORIDA, Tampa, FL (US)

(72) Inventors: Chris Peter Tsokos, Tampa, FL (US); Mohamed Ali M Abu Sheha, Tampa, FL (US)

(21) Appl. No.: 18/098,057

(22) Filed: Jan. 17, 2023

Related U.S. Application Data
(60) Provisional application No. 63/299,760, filed on Jan. 14, 2022.

(51) Int. Cl. A61B 5/00; G16H 50/20; G16H 50/30

(52) U.S. Cl. CPC A61B 5/4088; G16H 50/20; G16H 50/30

(57) ABSTRACT

In accordance with some embodiments, systems, methods, and media for predicting the conversion time of Mild Cognitive Impairment (MCI) to Alzheimer’s Disease (AD) in a patient are provided. In some embodiments, a system include a memory and a processor coupled to the memory. The processor is configured to: receive a plurality of risk factor indications and a plurality of interaction indications of a patient. Each interaction indication is an indication of interaction between two risk factor indications of the plurality of risk factor indications. The processor is further configured to obtain a trained machine learning model; apply the plurality of risk factor indications and the plurality of interaction indications to the trained machine learning model; and output a result based on the trained machine learning model.

### CROSS REFERENCE TO RELATED APPLICATIONS

This application claims the benefit of U.S. Provisional Patent Application Serial No. 63/299,760, filed Jan. 14, 2022, the disclosure of which is hereby incorporated by reference in its entirety, including all figures, tables, and drawings.

### BACKGROUND

Alzheimer’s is a devastating disease that gradually destroys a person’s memory. Alzheimer’s disease can develop slowly, and irreversibly. Eventually, Alzheimer’s can take away a person’s ability to carry out even simple daily life activities.

Based on data collected from the year 2000 to the year 2019, heart disease deaths decreased by 7.8%, while Alzheimer’s disease (AD) deaths increased by 146.2%. People who are medically classified with Mild Cognitive Impairment (MCI) may be treated and remain classified with MCI. Some people with MCI may revert back to Normal Cognition (NC). However, some people classified with MCI may develop Alzheimer’s disease. The length of a conversion time from MCI to Alzheimer’s disease may influence a person’s quality of life.

Accordingly, new systems, methods, and media for predicting a conversion time of Mild Cognitive Impairment to Alzheimer’s disease in patients are desirable.

### SUMMARY

In accordance with some embodiments of the disclosed subject matter, systems, methods, and media for predicting a conversion time of Mild Cognitive Impairment to Alzheimer’s disease in patients are provided.

In accordance with some embodiments of the disclosed subject matter, a system, method, apparatus, and non-transitory computer-readable medium for disease prediction are disclosed. The system includes a memory and a processor communicatively coupled to the memory. The memory stores a set of instructions which, when executed by the processor, cause the processor to: receive a plurality of risk factor indications and determine a plurality of interaction indications of a patient, each interaction indication of the plurality of interaction indications being an indication of interaction between at least two risk factor indications of the plurality of risk factor indications; obtain a trained machine learning model; apply the plurality of risk factor indications and the plurality of interaction indications to the trained machine learning model; and output a result based on the trained machine learning model.

In accordance with further embodiments of the disclosed subject matter, a system, method, apparatus, and non-transitory computer-readable medium for disease prediction model training are disclosed. The system includes a memory and a processor communicatively coupled to the memory. The memory stores a set of instructions which, when executed by the processor, cause the processor to: receive a plurality of sets of training data corresponding to a plurality of patients with Mild Cognitive Impairment (MCI); select, from each set of training data for a respective patient of the plurality of patients, a subset of the training data, the subset comprising: a plurality of risk factor indications and a plurality of interaction indications for the respective patient, the subset from each set of training data being a plurality of subsets of training data; obtain a plurality of ground truth conversion time indications corresponding to the plurality of patients; and train a machine learning model based on the plurality of subsets of training data, and the plurality of ground truth conversion time indications corresponding to the plurality of patients.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 illustrates a graph showing correspondence among three conventional cognitive dysfunction measures in Alzheimer’s disease.

FIG. 2 illustrates a schematic representation of an example dataset that may be used in accordance with some embodiments of the present disclosure

FIG. 3 illustrates an example graph comparing the differences in survival probability concerning gender according to some embodiments of the present disclosure.

FIG. 4 illustrates a scatter plot matrix, generated using mechanisms described herein, of target and continuous risk factors.

FIG. 5A illustrates a Q-Q plot for testing normality of time conversion according to some embodiments of the present disclosure.

FIG. 5B illustrates a Q-Q plot for testing normality of time conversion according to some embodiments of the present disclosure.

FIG. 6A illustrates an assessment of linear relationship assumptions of response variable and continuous risk factors, according to some embodiments of the present disclosure.

FIG. 6B illustrates a Q-Q plot generated in accordance with an example embodiment of the present disclosure.

FIG. 6C illustrates a scale-location plot that graphs the square root of standardized residuals with respect to fitted values, generated in accordance with an example embodiment of the present disclosure.

FIG. 6D illustrates an auto-correlation plot of residuals that graphs an auto-correlation function (ACF) of residuals with respect to lag, generated in accordance with an example embodiment of the present disclosure.

FIG. 7 shows an example ranking of risk factors according to some embodiments of the present disclosure.

FIG. 8 shows an example of a system for predicting a conversion time of Mild Cognitive Impairment to Alzheimer’s disease in a patient.

FIG. 9 shows an example of hardware that can be used to implement a computing device and a server, shown in FIG. 8 in accordance with some embodiments of the disclosed subject matter.

FIG. 10 shows an example of a process for predicting a conversion time of Mild Cognitive Impairment to Alzheimer’s disease in a patient in accordance with some embodiment of the disclosed subject matter.

FIG. 11 is a flow diagram illustrating an example process for disease prediction in accordance with some embodiment of the disclosed subject matter.

FIG. 12 is a flow diagram illustrating an example process for disease prediction model training in accordance with some embodiment of the disclosed subject matter.

The drawings illustrate only example embodiments and are therefore not to be considered limiting of the scope of the embodiments described herein, as other embodiments are within the scope of the disclosure.

### DETAILED DESCRIPTION

In accordance with various embodiments, mechanisms (which can, for example, include systems, methods, and media) for predicting a conversion time of mild cognitive impairment to Alzheimer’s disease in patients are provided.

Alzheimer’s disease is the leading cause of dementia. About 4.6 million new cases of dementia are predicted to occur every year, and the number of dementia cases are expected to double within the next ten years. Alzheimer’s disease is reported to be the sixth-leading cause of death in the United States. Mild Cognitive Impairment (MCI) represents a middle stage between Normal Cognition (NC) and Alzheimer’s disease (AD). Subjects with MCI are target groups for the detection, prognosis, and early treatment for AD. However, an individual’s diagnosis of MCI does not always convert to a diagnosis of Alzheimer’s. In a few cases, MCI might revert to the normal cognitive stage, or even remain stable.

Pathological development of Alzheimer’s disease could begin decades before warning signs and a clinical diagnosis. The pathological developments may include features such as clumps of Amyloid-beta (Aβ) plaques, intracellular neurofibrillary tangles (consisting of hyper-phosphorylated P-tau fibrils ruining our nerve cells), and loss of tiny gaps between nerve cells. Proteins that are tracked for pathological developments related to Alzheimer’s may be measured in picograms per milliliter (pg/ml) from cerebrospinal fluid.

The average healthy brain has more than 100 billion nerve cells recording recalled memories and telling our bodies what to do. In the brain of Alzheimer’s patients, it is believed that nerve cells may be slowly killed by plaques (e.g., Amyloid-Beta) and tangles (e.g., tau). The plaques may be clumps of amyloid protein pieces built up in the space between nerve cells, interrupting signals, and the tangles may be tau protein that have collapsed and twisted, building up inside the individual nerve cells, which may cause the tau protein to die. Thus, when the tau proteins die, irreversible changes may begin in the brain (e.g., pathological developments leading to Alzheimer’s disease). Therefore, it may be essential to investigate the plaques and tangles, how they interact with each other, develop over time in aging, and in early cognitive impairment stages.

MRI data may be used to detect brain changes caused by Alzheimer’s disease. Thus, an MRI scan may include volumetric measurements about a volume at a baseline (e.g., a measurement of an individual without an Alzheimer’s diagnosis) of one or more of the hippocampus, ventricles, entorhinal, fusiform, and intracranial volume (ICV). The memory area of a patient’s brain (e.g., the hippocampus, and other related structures in the temporal lobe) may be most vulnerable to the impact of Alzheimer’s in the brain. Thus, any effort to resist cognitive decline in a brain will be an outstanding achievement towards mitigating Alzheimer’s disease in society.

Apolipoprotein-E (APOE) 4 genotype may be a vulnerable gene for AD associated with increased deposition and decreased clearance of amyloid-beta. APOE has been shown to predict Mild Cognitive Impairment progression (MCI) to Alzheimer’s disease (AD). The APOE 4 genotype may be a factor in predicting a conversion time of MCI to AD in patients according to some embodiments of the present disclosure. Alternatively, the APOE 4 genotype may not be a factor of interest in predicting the conversion time of MCI to AD in patients according to some embodiments of the present disclosure. APOE 4 may be considered a potential confounder. Further, the APOE 4 genotype may be a factor with “non-carrier,” “heterozygous carrier,” and/or “homozygous carrier” levels.

There are several neuropsychological test measurements that the physician can use to evaluate a patient’s impaired memory, or thinking, according to mechanisms described herein. Some conventional methods to assess AD-related cognitive decline are the Alzheimer’s Disease Assessment Scale—Cognitive Subscale (ADAS-Cog), Mini-Mental State Examination (MMSE), and the Clinical Dementia Rating scale (CDR-SOB). However, the ADAS-Cog is more sensitive than the MMSE. The ADAS-Cog may be more reliable, less influenced by educational level, and less influenced by language skills than the MMSE.

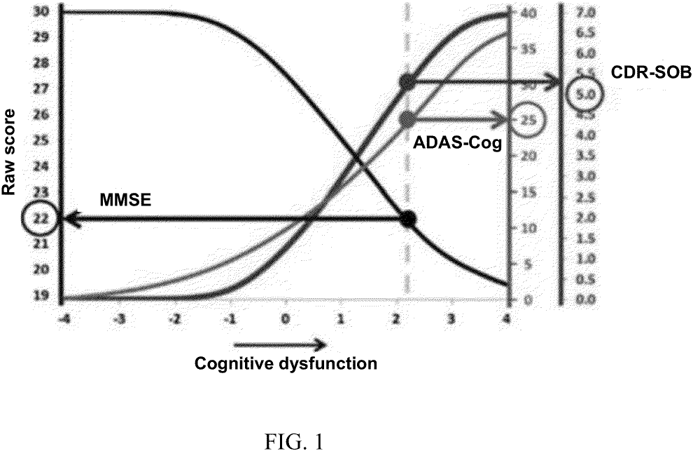

FIG. 1 illustrates a graph showing correspondence among three conventional cognitive dysfunction measures in Alzheimer’s disease. For example, at a cognitive dysfunction of about 2, a patient may have an MMSE score of about 22, an ADAS-Cog score of about 25, and a CDR-SOB score of about 5.

Statistical analysis may be performed to track progression of MCI patients based on if a patient converted or survived, as will be discussed further herein. Some mechanisms for predicting conversion time to AD may be focused on identifying predictors of conversion to Alzheimer’s disease (AD) in order to improve therapeutic strategies. For instance, the semi-parametric Cox regression analysis with baseline measures can be used to predict conversion time to AD. However, modeling based on the semi-parametric Cox regression analysis may assume that predictors remain constant over time, which may not be true. In other embodiments for predicting conversion time to AD, joint modeling of longitudinal and survival data may be used to assess the relationship between changes of measures and disease progression. Embodiments using such mechanism may have a significant improvement in the prediction of AD conversion among patients based on longitudinal change information, in addition to baseline. Additionally, baseline volumes of brain atrophy and their respective atrophy rates may significantly contribute to predicting the timing of earlier conversion from MCI to AD. Yearly change in volume may also be predictive, for example, when initial volumes are above a certain threshold.

Embodiments of the present disclosure may develop real data-driven analytical or predictive models, instead of considering conversion of MCI to AD as a binary response. For example, some embodiments of the present disclosure may assess various risk factors to predict the time to conversion (e.g., the time to conversion from MCI to AD) dependent on baseline measures using supervised machine learning methods.

Models generated using mechanisms described herein may fulfill modeling assumptions such as linearity, multicollinearity, and normality assumption related to errors and responses. Furthermore, models generated using some mechanism described herein may be validated (e.g., for precision, and accuracy) using statistical evaluations such as, for example, R square (R²), R square adjusted (Radj2), predicted (R²), and residual analysis.

Mechanisms described herein may identify individual risk factors that significantly contribute to the conversion time of MCI to AD in patients. Further, mechanisms described herein may identify the interactions among risk factors that significantly contribute to the conversion time of MCI to AD in patients. Mechanisms described herein may rank the individual risk factors and interaction with respect to their amount of contribution to the conversion time of MCI patients to AD. Mechanisms described herein may be used to generate and/or train models (e.g., artificially intelligent models, machine learning models, or statistical analysis models) that predict the conversion time of MCI to AD in patients with a relatively high degree of accuracy. Some models generated using mechanisms described herein may predict the conversion time of MCI to AD in patients with an accuracy of 93.5%. Furthermore, mechanisms described herein may be evaluated via statistical methods to validate results and ensure a relatively high degree of accuracy.

Some embodiments of the present disclosure may aggregate baseline data using databases formulated via testing, clinical trials, or surveys. Alternatively, some embodiments of the present disclosure may aggregate baseline data using publicly available databases, such as, for example, the Alzheimer’s Disease Neuroimaging Initiative database (ADNI). For example, some databases for tacking the progression of Alzheimer’s disease, such as the ADNI, may include biospecimens, genetic, magnetic resonance imaging (MRI), positron emission tomography (PET), and clinical information of individuals.

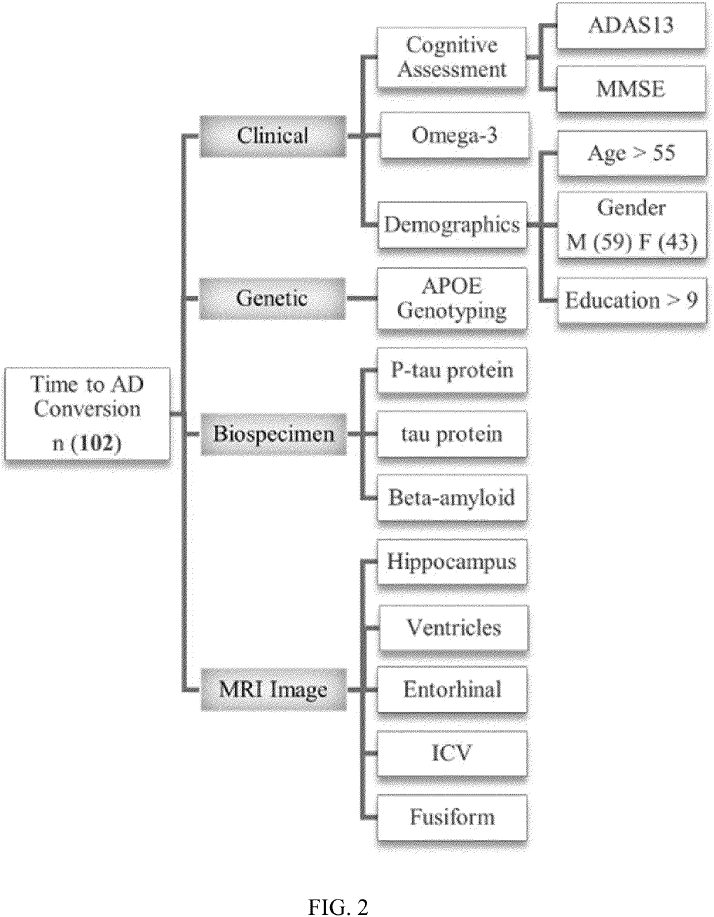

FIG. 2 illustrates a schematic representation of an example dataset that may be used in accordance with some embodiments of the present disclosure. The example dataset includes information from 102 individuals, including their respective conversation times to AD. The example dataset includes clinical information. The clinical information may include cognitive assessment. The cognitive assessment may include ADAS13, and MMSE. The clinical information may further include omega-3, and demographics. The demographics may include one or more from the group of: if the education of an individual is greater than nine years, gender, and if the age of an individual is greater than 55 years. The example dataset further includes genetic information. The genetic information may include APOE genotyping. The example dataset further includes biospecimen. The biospecimen may include one or more from the group of: p-tau protein, tau protein, and Beta-amyloid. The example dataset further include MRI images. The MRI images may include one or more from the group of: hippocampus, ventricles, entorhinal, ICV, and fusiform.

Some embodiments of the present disclosure may generate and/or train one or more models by analyzing subjects enrolled in the ADNI with complete data recorded for 14 concomitant risk factors, and a clinical diagnosis for each visit (e.g., NC, or MCI). Furthermore, some embodiments of the present disclosure may exclude subjects (e.g., individuals, or patients) diagnosed with AD at a baseline analysis. A response variable may be a recording time of an individual’s conversion from an MCI diagnosis to AD within an 8 year follow-up.

In some embodiments of the present disclosure, data collected from patients, such as the data collected in FIG. 2 from 102 patients, may be used to generate and/or train a machine learning analytical or predictive model. Alternatively, in some embodiments of the present disclosure, data collected from patients may be used to generate linear analytical or predictive models, or non-linear analytical or predictive models. In some embodiments, one may apply regression techniques, graphing techniques, inductive reasoning approaches, or other artificial intelligence evaluations to generate a model for predicting conversion time of MCI to AD in patients.

In one example embodiment, a multivariate linear regression (MLR) model can describe the relationships between a continuous outcome (response variable) and a set of covariates (risk factors). The response variable may be defined as a function of the risk factors (e.g., the risk factors of FIG. 2 discussed earlier herein) and all possible interactions. The general structure of the model with all possible interactions and additive error structure could be expressed as follows:

$$T i m e = {β}_{0} + {\sum }_{i} {α}_{i} {x}_{i} + {\sum }_{j} {γ}_{j} {k}_{j} + {ε}_{i} ,$$

where β₀ is the intercept of the model, αᵢ is the coefficient of i^{th} individual risk factor xi, γⱼ is the coefficient of j^{th} interaction term kⱼ, and εᵢ denotes the random disturbance or residual error of the model. For examples, in the two-predictor case (i.e., xi and x₂), the two-way interaction term (k₁₂) is constructed by computing the product of xi and x₂. Therefore, the algorithm will build a matrix with four columns for β₀, α₁, α₂, γ₁₂ respectively.

In the statistical model structure for multivariate linear regression, there may be several assumptions. Some embodiments of the present disclosure may generate a predictive model based on one or more assumptions from the group of: linearity, normality of residuals, homoscedasticity, no autocorrelation, and multicollinearity. Some embodiments of the present disclosure may rely on no assumptions to generate a predictive model.

Some embodiments of the present disclosure may use an ordered quantile transformation method to assist in generating the predictive model disclosed herein. The ordered quantile (ORQ) normalization method was initially proposed by Bartlett in 1947, and further developed in 1952 by Van der Waerden. A quantile transform may be used to map a probability distribution of a given variable to another probability distribution, such as a uniform or normal probability distribution. The quantile transformation function may be defined by Equation 2 below, where x represents the original data, φ denotes the standard normal cumulative distribution function (cdf), rank(x) is the rank of each observation, and length(x) refers to the number of observations.

$$g \left(x\right) = {ϕ}^{- 1} \left(\frac{R a n k \left(x\right) - 0.5}{l e n g t h \left(x\right)}\right) ,$$

ORQ normalization may be applicable across a variety of probability distributions. ORQ normalization can successfully convert right/left-skewed data, and multimodal data into a vector that follows a normal (Gaussian) distribution. Therefore, ORQ normalization can be used in accordance with mechanisms described herein to normalize data sets, such as the data set in FIG. 2, prior to analysis performed to generate a predictive model (e.g., a machine learning model, or another model, to predict the conversion time of MCI to AD in a patient).

Conventional studies have shown that the Alzheimer’s disease (AD) incidence is higher in women than in men without identifying any reason for why. Thus, some embodiments of the present disclosure analyze if there is a difference in the conversion time (survival probabilities) of males and females diagnosed with MCI. Using the log-rank test from the Kaplan-Meier non-parametric test, the differences in conversion times of males and females can be compared.

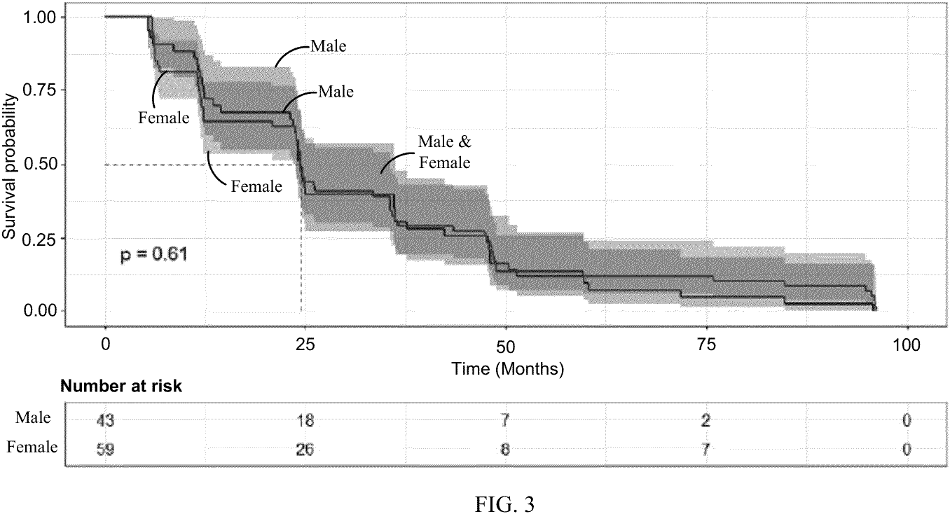

FIG. 3 illustrates an example graph comparing the differences in survival probability concerning gender according to some embodiments of the present disclosure. Specifically, FIG. 3 shows the log-rank test result with p-value=0.61, which indicates a failure to reject the null hypothesis. Thus, as shown in FIG. 3, there is no difference in the conversion time probabilities concerning gender (e.g., biological genders, such as, male, or female). The number of males and females who were at risk of conversion to AD within each follow-up time from the example data set of FIG. 2, were included in FIG. 3, as well. Some embodiments of the present disclosure used to predict MCI conversion time can be focused on conducting a survival analysis of patients diagnosed with MCI. Some embodiments of the present disclosure may rely on the findings of FIG. 3 that gender is not a bias in predicting the conversion time of MCI to AD in patients.

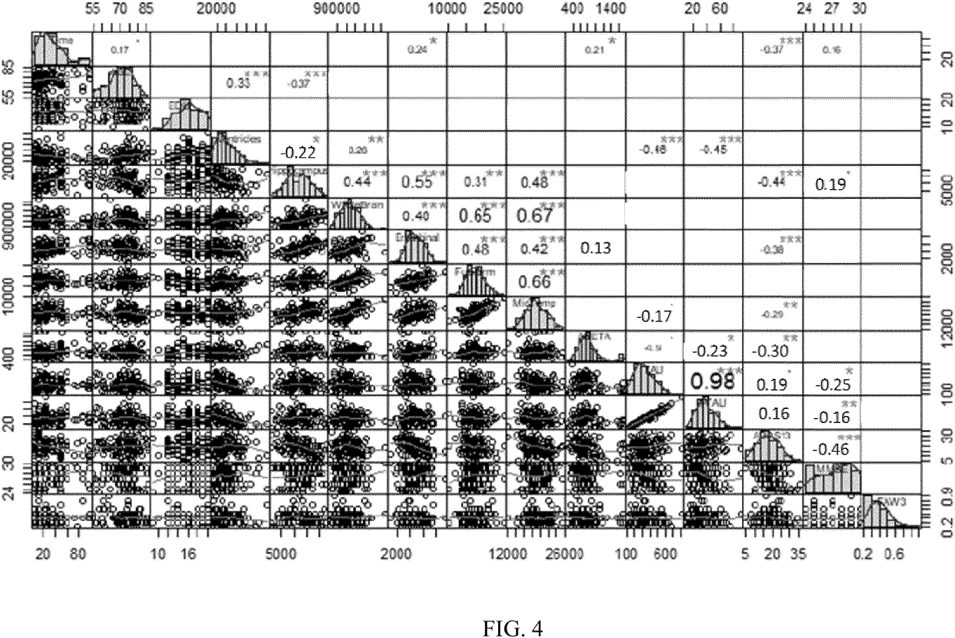

FIG. 4 illustrates a scatter plot matrix, generated using mechanisms described herein, of target and continuous risk factors. Generally, embodiments of the present disclosure may use one or more scatter plot matrices to visually assess the relationship between the target variable (Time) and any of the continuous risk factors, especially the continuous risk factors that may seem suspicious. With regard to the example embodiment used to generate FIG. 4, a strong positive association between the variables tau and P-tau may be observed, while the remaining continuous risk factors show a moderate association therebetween. However, in some embodiments of the present disclosure, multicollinearity can be tested using the variance inflation factor (VIF) after a regression analysis is conducted.

Still referring to the specific example matrix of FIG. 4, the target variable’s distribution is skewed (i.e., the distribution is pushed to the far left of the matrix). Most of the risk factors have an approximate normal distribution. However, logarithmic transform can be used to make the left-skewed risk factors more Gaussian. For example, in some embodiments of the present disclosure, logarithmic transform can be used to make the distribution of Ventricles, Fusiform, and FAW3 more Gaussian. It is contemplated that logarithmic transform can be used to make other disclosed risk factors of the present disclosure more Gaussian, as well.

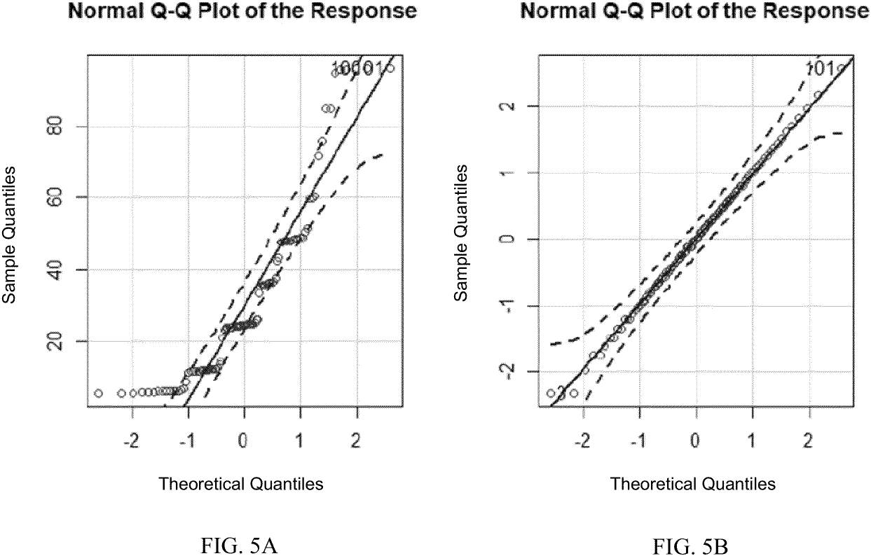

FIGS. 5A and 5B illustrate Q-Q plots for testing normality of time conversion according to some embodiments of the present disclosure. In the example embodiment of FIG. 5A, evidence is shown of a violation from normality in the response’s original data. One of the underlying assumptions for developing the statistical model for some embodiments of the present disclosure may be that the response variable should follow the Gaussian probability distribution. Thus, an ORQ transformation may be used to map the response distribution normally, which may improve the modeling performance, and the relationship with the input risk factors. FIG. 5B illustrates the data of FIG. 5A after an ORQ transformation has been applied. Therefore, some embodiments of the present disclosure support that a transformed response distribution can follow a Gaussian probability distribution after an ORQ transformation has been applied. Also, normality evidence can be supported by a goodness of fit testing (Shapiro-Wilk normality test), given a significant p-value of 0.91.

Some embodiments of the present disclosure may start with a full statistical model, including risk factors. For example, an example embodiment may consider fourteen risk factors (e.g., the fourteen risk factors of FIG. 2: ADAS13, MMSE, age > 55, gender, education > 9, APOE genotyping, p-tau protein, tau protein, beta-amyloid, hippocampus MRI image, ventricles MRI image, entorhinal MRI image, ICV MRI image, and fusiform MRI image), and all possible interactions therebetween that may significantly contribute to conversion time. Thus, initially, the example embodiment with fourteen risk factors may structure a model with 91 total terms that include the primary contribution of attributable variables and every possibility of two-way interactions.

To create an accurate predictive model, some embodiments of the present disclosure may receive a dataset, split the dataset in two, and use 80% of the dataset to fit (train) the model in a supervised learning manner. The embodiments may then use the remaining 20% of the data set to evaluate (test) the trained model. Then, the embodiments may perform a model selection process (e.g., selecting the most accurate model to be used for predictive analysis). Some embodiments may use a backward feature selection method to perform the model selection.

A concern of generating a trained model is that a model will be overfit to the trained data, and therefore may be inaccurate for other datasets that may be evaluated using the trained model. Training a model with too many attributable variables can lead to the model being overfit to the training data and produce a poor prediction when applying the analytical model to received data in a real-world environment (e.g., confidential information in a lab, clinic, or consumer environment). Consequently, embodiments of the present disclosure may use feature selection methods to avoid overfitting a model, by using only a set of the risk factors that contribute to the model. In some examples, the set of the risk factors may be factors that most contribute to the conversion time of MCI to AD. For example, some embodiments of the present disclosure may generate models using the one risk factor, or the two risk factors, or the three risk factors, or the four risk factors. The selected risk factors may be the factors that are found to have the greatest impact on the conversion time from MCI to AD in a patient.

Furthermore, in some embodiments of the present disclosure, stepwise backward elimination is used because it gives less bias mean square error (MSE) values and deals with overfitting the model, which can be important for the model’s prediction efficiency. However, some embodiments of the present disclosure selected a model based on the Akaike information criterion (AIC) (e.g., smallest AIC).

Additionally, embodiments of the present disclosure may train models using repeated cross-validation. Some cross validation techniques applied using mechanisms described herein use 10-fold cross-validation. Further, the cross-validation may be repeated three times wherein each of the repetition folds are split differently between one another. In 10-fold cross validation, the training set is divided into ten equal subsets. One of the ten equal subsets is taken as a testing or validation set and the remaining nine subsets are taken as a training set to generate the trained model that can predict the conversion time of MCI to AD in patients. After each repetition of the cross validation, a model assessment metric can be computed. The model assessment metric may be computed using Equation 3 below.

$$R M S E = \sqrt{\frac{{\sum }_{i = 1}^{n} {\left({y}_{i} - {\hat{y}}_{i}\right)}^{2}}{n}} .$$

The validation set (i.e., the hold-out set from the training model) may be used to calculate an initial estimate of the trained model’s performance. The initial estimate can provide a reasonable assessment of a models skills (e.g., precision, or accuracy), in comparison to a single train-test split.

However, given that the model selection method and criterion of choosing a good model (e.g., a model with accurate results), may be based on the coefficient of determination, e.g., R², which is 0.944, and adjusted R² of 0.939, respectively. R² may be a criteria for evaluating fit and quality of a model generated using mechanisms disclosed herein. A ratio of adjusted R-squared to R-squared of 0.995 may represent a decrease in a proposed model’s fit (e.g., goodness fit) when the model is applied to new data set. Generally, when the ratio of adjusted R-squared to R-squared is relatively higher, the fit and quality of the model is relatively better.

Therefore, statistical analysis estimation performed in accordance with some embodiments of the present disclosure may determine that ten out of fourteen risk factors (e.g., the fourteen risk factors disclosed earlier herein with respect to FIG. 2) at baseline may significantly contribute to the conversion time from MCI to AD in patients. Namely, Age, Education, Ventricles, Hippocampus, Entorhinal, Fusiform, Amyloidbeta, Tau, pTau and ADAS13 may be found to significantly contribute to the conversion time. Further, four interaction terms, namely, Abeta ∩ Tau, Hippocampus ∩ pTau, Ventricles ∩ pTau, and Hippocampus ∩ Educational may be found to significantly contribute to the conversion time.

Thus, in a preferred embodiment of the present disclosure, the proposed analytical model is based on ten significant risk factors and four interaction terms to accurately estimate the time-to-conversion of mild cognitive impairment (MCI) patients to AD patients. Equation 4 below may give the analytical form of the model generated in the preferred embodiment.

$$\begin{matrix}T \hat{ι} m e = - 0.003 + 0.062 \times A g e - 0.0098 \times E d u c a t i o n a l + 0.058 \times \\ V e n t r i c l e s - 0.079 \times H i p p o c a m p u s + 0.046 \times E n t o r h i n a l - 0.024 \times \\ F u s i f o r m + 0.085 \times A b e t a - 0.12 \times T a u + 0.34 \times p T a u + 0.025 \times \\ A D A S 13 - 0.012 \times V e n t r i c l e s ⋂ p T a u + 0.0073 \times H i p p o c a m p u s ⋂ \\ E d u c a t i o n a l - 0.69 \times A b e t a ⋂ T a u - 0.0045 \times H i p p o c a m p u s ⋂ p T a u .\end{matrix}$$

The estimated conversion time (Tîme) may be based on ordered quantile transformation data from Equation 2. Therefore, some embodiments of the present disclosure may use an anti-ordered quantile transformation to transform back to an actual estimate of the conversion time from MCI to AD. As a result, if a new patient is diagnosed with MCI, then, given the values of the significant risk factors identified in Equation 4, some embodiments of the present disclosure can generate an analytical or predictive model to accurately estimate the conversion time to Alzheimer’s disease in a patient.

In some embodiments of the present disclosure, after performing a multivariate linear regression, several assumptions may be checked. The assumption may include one or more from the group of: linearity, normality of residuals, homoscedasticity of residuals, no autocorrelation, and no perfect multicollinearity among risk factors.

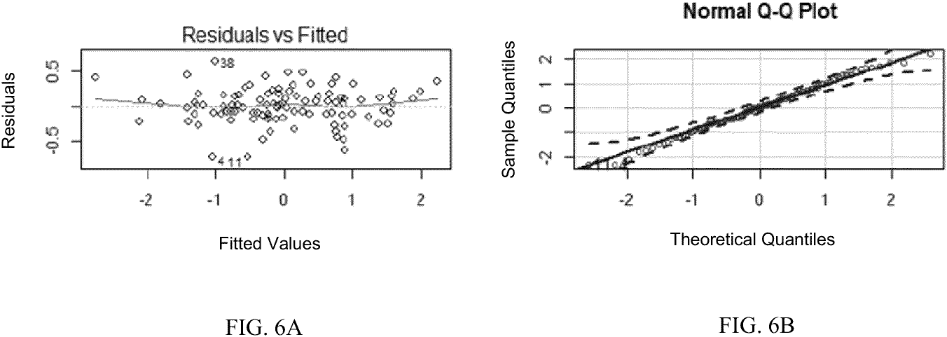

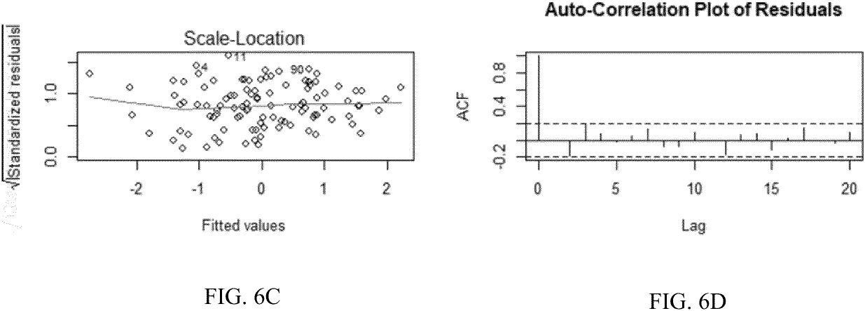

FIGS. 6A-6D illustrates residual diagnostic plots according to some embodiments of the present disclosure. FIG. 6A illustrates an assessment of linear relationship assumptions of the response variable and the continuous risk factors. FIG. 6B illustrates a Q-Q plot generated in accordance with an example embodiment of the present disclosure. The residual analysis shown in the example Q-Q plot of FIG. 6B justify the model assumptions of normality and constant error variance. FIG. 6C illustrates a scale-location plot that graphs the square root of standardized residuals with respect to fitted values, generated in accordance with an example embodiment of the present disclosure. FIG. 6D illustrates an auto-correlation plot of residuals that graphs an auto-correlation function (ACF) of residuals with respect to lag, generated in accordance with an example embodiment of the present disclosure.

In an example embodiment of the present disclosure, a structured analytical model for predicting conversion time may have a mean residual equal to 3.1 × 10⁻⁶ (e.g., almost or approximately zero). The variance of the residual may be 0.07, the standard deviation may be 0.21, and the standard error of the residuals may be 0.23. The statistics of the example embodiment support that the structured analytical model is high quality. Furthermore, proof of normality may be supported by Shapiro Wilk’s test of the normal probability distribution, given by a high p-value of 0.91.

Analytical or predictive models generated in accordance with some embodiments of the present disclosure may perfectly satisfy an assumption of homogeneity (constant variance) of residuals. For example, FIG. 6C indicates homogeneity for an example plot of residual data by illustrating a horizontal line with equally spread points. Furthermore, some embodiments of the present disclosure may test model residuals to be independent and uncorrelated. For example, in the example plot of FIG. 6D generated in accordance with an embodiment of the present disclosure, the autocorrelation plot at lag 0 has a correlation of 1 i.e., the data may be correlated with itself. Since the correlation (Y-axis) from the following immediate line onwards would drop to a near zero value below the dashed line, it implies that the means of the residuals were not auto-correlated. A formal test (Durbin Watson=1.97) for autocorrelation on the example plot of FIG. 6D may reveal a p-value of 0.472, which strongly supports that no autocorrelation is present.

To enhance the reliability of the testing outcomes, some embodiments of the present disclosure may use one or more trained models to predict the time-to-conversion from MCI to AD using one or more risk factor test datasets. The performance of the trained analytical model may then be reported based on a predictive R² and a root mean square error (RMSE). The RMSE measures the difference between the observed value and predicted values. The smaller the RMSE value, the closer the predicted conversion time is to the actual conversion time, and the more accurate the model prediction. An example embodiment of the present disclosure may include a proposed model that has an RMSE value of 0.23, indicating a very good predictive accuracy.

Generally, R² and adjusted R² may be measures of a percentage of variation in conversion time that a model explains; nevertheless, the predicted R² may be used to determine how well a model predicts the conversion time for new observations. However, the predicted R² may be calculated using the predicted residual sums of squares (PRESS), as shown in Equation 5 below, where SST is the sums of squares total.

$$P r e d i c t i v e {R}^{2} = \left[1 - \left(\frac{P R E S S}{S S T}\right)\right] \times 100 .$$

Embodiments of the present disclosure may include methods for ranking individual risk factors, and interaction risk factors. In some embodiments of the present disclosure, the R² criteria may be used to rank an individual (e.g., a patient) along with the significant interaction risk factors with respect to the percent of contribution of predicting the conversion time.

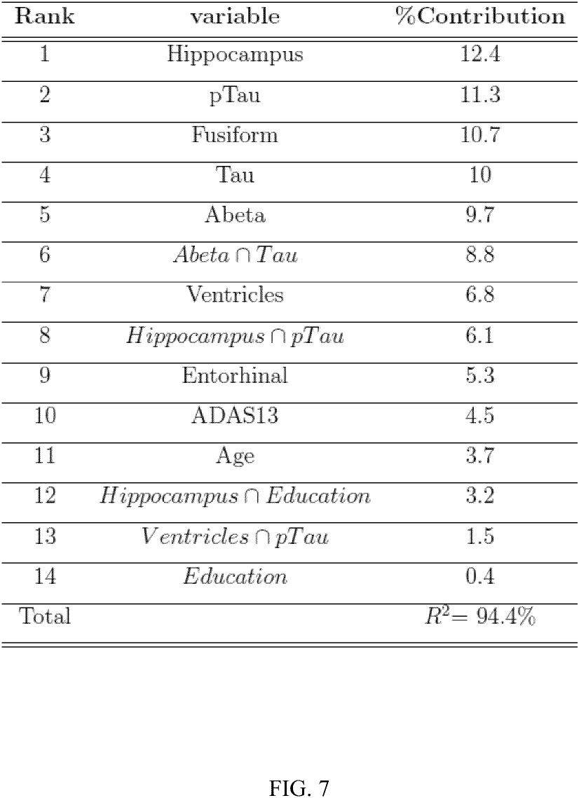

FIG. 7 shows an example ranking of risk factors according to some embodiments of the present disclosure. The risk factors are listed long with their percent of overall contribution to predicting conversion time of MCI to AD in patients. The risk factor that has the most significant contribution to the conversion time in MCI patients is the Hippocampus, which contributes 12.4% of the conversion time. Again, the Hippocampus has interacted with pTau and Education, which contribute 6.1% and 3.2%, respectively, increasing the Hippocampus’ contribution to the conversion time of MCI to AD.

In the example ranking of FIG. 7, the next largest contribution is pTau with 11.2% contribution; its interaction with the hippocampus and ventricles increases its importance in predicting the conversion time with an overall percentage contribution of 18.9%. Fusiform was ranked as the third contributor to the conversion time, followed by Tau and Abeta. Even though Fusiform ranked third, Tau and Abeta are considered the most significant to the conversion time. Tau and Abeta have an overall percentage contribution of 18.8% and 19.7% (e.g., by combining the individual and related percent contributions), respectively, while Fusiform contributed 10.7%.

However, in the example ranking of FIG. 7, Education has the least contribution to the conversion time of patients diagnosed with MCI among the significant risk factors. Therefore, summing all of the risk factors in FIG. 7 together totals a 94.4% contribution to the conversion time. An analytical or predictive model generated using embodiments of the present disclosure that includes the risk factors of FIG. 7 may predict a conversion time from MCI to AD in a patient that is 94.4% accurate. Further the predicted conversion time may have an R squared adjusted value of 93.9%, and a predictive R² value of 93.5%. Analytical or predictive models generated using embodiments of the present disclosure may be highly accurate models for predicting conversion time from MDI to AD.

Some embodiments of the present disclosure generate real data-driven analytical or predictive models that may identify ten significant risk factors and four interactions that significantly contribute to predict the time of conversion to AD from MCI in patients. Some embodiments of the present disclosure also examine the quantitative effect that measurements of cerebrospinal fluid (CSF) biomarkers, magnetic resonance imaging (MRI), and clinical/psychometric assessments have on the conversion time.

Some proposed analytical models generated using methods described herein predict the conversion time with a very high degree of accuracy. For example, the predictive accuracy may be 93.5%. Mechanisms described herein may be used to statistically validate results of the model using various methods to attest to its high quality. Methods, system, and media disclosed herein for predicting the conversion time of MCI to AD in patients may offer essential and valuable findings for patients. For example, given any set of observations of identified significant risk factors, some embodiments disclosed herein can obtain an excellent prediction of the conversion time to the AD of patients diagnosed with MCI. Some embodiments of the present disclosure may identify the individual risk factors and interactions that contribute significantly to the conversion time to the AD of patients diagnosed with MCI. Some embodiments of the present disclosure may rank the risk factors based on the contribution to the conversion time to the AD of patients diagnosed with MCI. Some embodiments of the present disclosure can perform surface response analysis to identify each risk factor’s contribution to maximize the conversion time to the AD of patients diagnosed with MCI. Some embodiments of the present disclosure can compute confidence interval for estimated and predicted values of conversion times.

The results (e.g., a predicted conversion time) obtained from models generated using mechanisms described here may help evaluate the efficacy and safety of upcoming treatment for mild cognitive impairment patients. These markers may improve the identification of subjects with MCI who are at risk for Alzheimer’s disease. Therefore, if users of methods, systems, and media disclosed herein can delay the onset of cognitive impairment (e.g., forgetfulness) by maximizing the conversion time (e.g., by adjusting, or otherwise addressing risk factors contributory to conversion time), then this would be a dramatic improvement in people’s quality of life.

Accordingly, the embodiments disclosed herein are practical, accurate, and effective for predicting the conversion time of Mild Cognitive Impairment (MCI) to Alzheimer’s disease (AD) in patients.

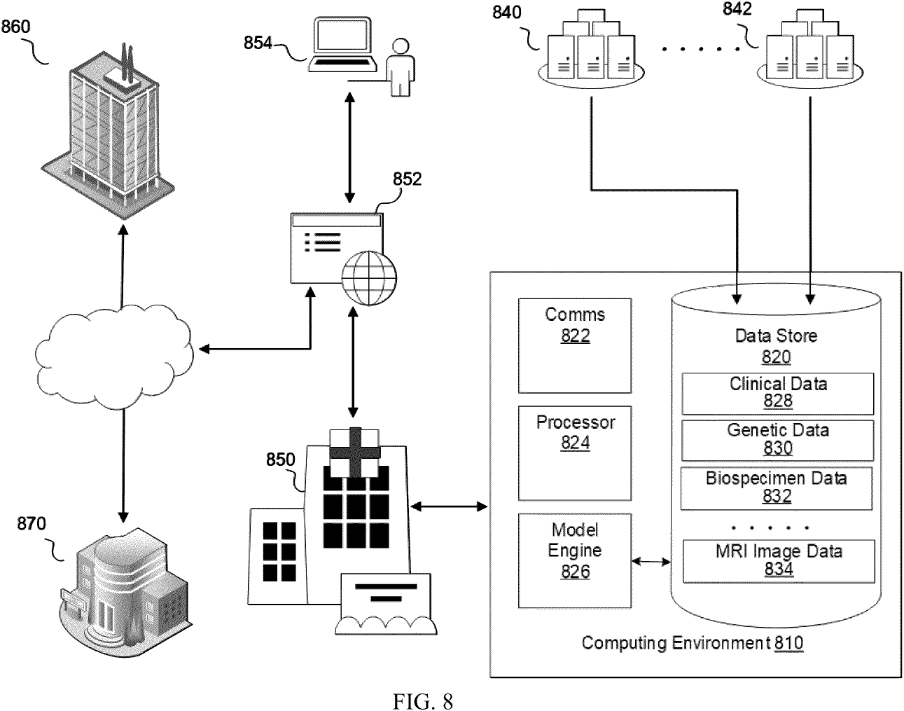

FIG. 8 shows an example of a system for predicting the conversion time of Mild Cognitive Impairment (MCI) to Alzheimer’s disease (AD) in patients in accordance with some embodiments of the disclosed subject matter. A computing environment 810 comprises a data store 820, a communications connection 822, at least one processor 824, and a model engine 826. The computing environment 810 may be implemented via computing resources of a company or institutional network (e.g., local servers, company network) or may be implemented via a cloud computational resource (e.g., one or more remote servers). The communications connection 822 may be a suitable connection for allowing the computing environment to communicate with remote resources and users, such as any suitable Internet connection or LAN/WAN connection. The computing environment 810 can be coupled via communications connection 822 to one or more networks embodied by the Internet, intranets, extranets, wide area networks (WANs), local area networks (LANs), wired networks, wireless (e.g., cellular, 802.11-based (Wi-Fi), Bluetooth, etc.) networks, cable networks, satellite networks, other suitable networks, or any combinations thereof. The computing environment 810 can communicate with other computing devices and systems using any suitable systems interconnect models and/or protocols. The computing environment 810 can be coupled to any number of network hosts, such as website servers, file servers, network switches, networked computing resources, databases, data stores, and other network or computing platforms. In the illustrated embodiment, the computing environment is in direct communication with a facility database or data store 820.

Processor 824 may comprise a one or more processors of local servers, or may be implemented as a virtual processor/virtual machine. Processor 824 may include or be connected to a memory 826 that stores software instructions which cause the processor to execute and operate an application that implements the algorithms and techniques described herein. For example, memory 826 may comprise instructions implementing a model engine for predicting the conversion time of MCI to AD in a patient, as described herein, as well as executing the various communication and operational tasks described herein. The model engine 826 may be configured to develop and analyze the predictive models described herein. In one example, the model engine 826 is configured to identify a plurality of risk factors (e.g., the risk factors discussed above with respect to FIG. 2) and a corresponding percent contribution that quantifies the impact that each risk factor has in predicting a conversion time from MCI to AD in a patient. The model engine 826 can also be configured to rank the plurality of risk factors from most to least contributory, and output a recommendation for treatment options to extend a conversion time from MCI to AD, among other functions described herein. Additionally or alternatively, the model engine stored in memory 826 and implemented by processor 824 can process data for a patient, for example clinical data, genetic data, biospecimen data, or MRI images, etc. and provide specific percent contributions of each category of data to indicate a predicted conversion time from MCI to AD in a patient.

Data store 820 may be a large database comprising data utilized for a variety of purposes, implemented via local network storage (e.g., on-site at a facility, research institution, or hospital as shown in FIG. 8) or cloud storage. Data store 820 may be in communication with one or more remote servers 840-42, in which case the computing environment simply retrieves data on an as-needed basis from the remote data services 840-42. Data store 820 may comprise data representing the risk factors for an MDI diagnosis converting to an AD diagnosis, as described herein. The data may include clinical data 828 (e.g., cognitive assessments, Omega-3, demographics); genetic data 830 (e.g., APOE genotyping); biospecimen data 832 (e.g., a quantity of p-tau protein, tau protein, or beta-amyloid); MRI image data 834 (e.g., MRI images of a hippocampus, ventricles, entorhinal, ICV, or fusiform), and other information as described above. In one example, the data store can be the Alzheimer’s Disease Neuroimaging Initiative (ADNI) database. In some embodiments, the data store may be an aggregation of data obtained from research institutions, or hospitals, or consumers of a product (e.g., a mobile application, a desktop application, or an online interface) that collects data regarding individuals with MCI.

The computing environment 810 can be implemented so as to perform one or more of a variety of functions for different types of users, through implementation of the models and algorithms described above. For example, the system 810 can provide services to a facility, clinic, or hospital 850. In such an embodiment, the system 810 may implement a website, interface, or user portal 852 accessible by the user. The facility, hospital, or other user 850 may upload certain data regarding one or more patients with MCI. Then the system 810 may process the data using the model engine 826 to determine a predicted conversion time from MCI to AD in a patient, and a set of rankings corresponding to the contributory risk factors in the conversion of MCI to AD in the patient (e.g., the risk factors with a higher contributory percentage relative to other risk factors may be ranked higher than the other risk factors on a list). Further, the model engine 826 may make recommendations for the patient regarding treatment plans for extending the conversion time from MCI to AD based on the ranking of contributory risk factors.

The website, interface, or user portal 852 may be an interface found on a computer in the hospital 950. Alternatively, or additionally, the website, interface, or user portal 852 may be an interface found on a home computer or similar computing device that allows a user 854 to upload information that may be stored in the data store, and subsequently receive a predicted conversion time from MDI to AD in a patient, as described herein. Alternatively, or additionally, the website, interface, or user portal 852 may be an application found on a mobile or wearable device that allows a user 854 to input information corresponding to risk factors for AD. Subsequently, the user 854 may receive, via the mobile application or wearable device application, an output that includes a predicted conversion time from MDI to AD in a patient, as described herein.

In one further embodiment, the foregoing techniques may be used by insurers, social service planning commissions, or healthcare organizations to predict and manage future expected outcomes for patients with mild cognitive impairment. For example, patients enrolled in specific health plans may be asked to submit information (pertinent to the risk factors described herein) to assist such entities in calculating time to onset of full AD. Using this tool, a software application could provide plans for ensuring suitable housing and care will be available in the future for each community or healthcare organization.

The contributions of the individual risk factors and interactions between the individual risk factors may be determined using analytical techniques that are readily apparent to one of ordinary skill in the art. For example, one may perform principal component analysis or singular value decomposition to determine the relation between one or more risk factors and subsequently calculate the respective contributions. Further, in some embodiments, one can train a machine-learning model, such as a neural network to determine the relation between one or more risk factors and subsequently calculate the respective contributions. In other embodiments, one may apply regression techniques, graphing techniques, inductive reasoning approaches, or other artificial intelligence evaluations to determine the relation between one or more risk factors and subsequently calculate the respective contributions.

Alternatively, or in addition to the foregoing operation, the system could also provide services to research firms and other businesses 860 focused on predicting conversion times from MCI to AD, or developing therapies and/or treatments for Alzheimer’s disease based on contributing risk factors. For example, the research firm 860 may use the model engine 826 of the hospital 850 to build and validate a predictive model for a specific treatment that seeks to address one or more contributory risk factors in one or more patients. The model building may proceed using the steps described herein above.

Additionally, the system 810 could also be utilized by various governmental agencies, regulatory bodies, insurance companies, or the like 870. For example, an evaluation of contributory risk factors to Alzheimer’s disease could help to target funding (e.g., grants) into the research of treatments or therapies directed toward specific risk factors found to be contributory to a significant number of patients with Alzheimer’s disease.

The computing systems and devices of environment 810 can be located at a single installation site or distributed among different geographical locations. The computing devices in such networks can also include computing devices that together embody a hosted computing resource, a grid computing resource, and/or other distributed computing arrangement.

Furthermore, mechanisms described herein may be used to evaluate the effectiveness of one or more drugs used to treat Alzheimer’s (e.g., by evaluating how successful a drug is at slowing down or stopping the conversion of Mild Cognitive Impairment to Alzheimer’s disease in a patient). For example, mechanisms described herein may be used to evaluate the effectiveness of a drug (e.g., a drug used to treat Alzheimer’s) approved by the United States Food and Drug Administration, such as, for example, Aduhelm®. Individuals (e.g., doctors, nurses, lab technicians, or users 854), insurance companies, research firms (e.g., research firm 860), government entities (e.g., government entity 870), businesses, or other organizations may use mechanisms described herein to evaluate whether a drug (e.g., Aduhelm®) is effective at increasing a predicted conversion time of Mild Cognitive Impairment to Alzheimer’s disease in a patient (e.g., slowing, or eliminating, the onset of Alzheimer’s in the patient). Alternatively, or additionally, individuals (e.g., doctors, nurses, lab technicians, or users 854), insurance companies, research firms (e.g., research firms 860), government entities (e.g., government entity 870), businesses, or other organizations may use mechanisms described herein to evaluate whether a drug decreases a predicted conversion time of Mild Cognitive Impairment to Alzheimer’s disease in a patient (e.g., progressing the onset of Alzheimer’s in the patient). The effectiveness of a drug on a predicted conversion time of Mild Cognitive Impairment to Alzheimer’s disease in a patient may impact a recommended course of treatment for the patient.

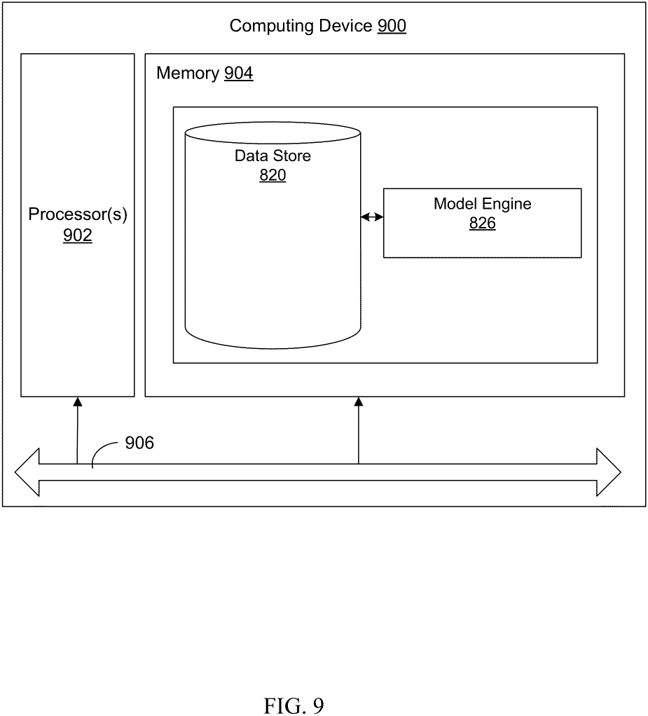

FIG. 9 illustrates an example schematic block diagram of a computing device 900 for the computing environment 810 shown in FIG. 8 according to various embodiments described herein. The computing device 900 includes at least one processing system, for example, having a processor 902 and a memory 904, both of which are electrically and communicatively coupled to a local interface 906. The local interface 906 can be embodied as a data bus with an accompanying address/control bus or other addressing, control, and/or command lines.

In various embodiments, the memory 904 stores data and software or executable-code components executable by the processor 902. For example, the memory 904 can store executable-code components associated with the model engine 826 for execution by the processor 902. The memory 904 can also store data such as that stored in the data store 820, among other data.

It is noted that the memory 904 can store other executable-code components for execution by the processor 902. For example, an operating system can be stored in the memory 904 for execution by the processor 902. Where any component discussed herein is implemented in the form of software, any one of a number of programming languages can be employed such as, for example, C, C++, C#, Objective C, JAVA®, JAVASCRIPT®, Perl, PHP, VISUAL BASIC®, PYTHON®, RUBY, FLASH®, or other programming languages.

As discussed above, in various embodiments, the memory 904 stores software for execution by the processor 902. In this respect, the terms “executable” or “for execution” refer to software forms that can ultimately be run or executed by the processor 902, whether in source, object, machine, or other form. Examples of executable programs include, for example, a compiled program that can be translated into a machine code format and loaded into a random access portion of the memory 904 and executed by the processor 902, source code that can be expressed in an object code format and loaded into a random access portion of the memory 904 and executed by the processor 902, or source code that can be interpreted by another executable program to generate instructions in a random access portion of the memory 904 and executed by the processor 902, etc.

An executable program can be stored in any portion or component of the memory 1004 including, for example, a random access memory (RAM), read-only memory (ROM), magnetic or other hard disk drive, solid-state, semiconductor, universal serial bus (USB) flash drive, memory card, optical disc (e.g., compact disc (CD) or digital versatile disc (DVD)), floppy disk, magnetic tape, or other types of memory devices.

In various embodiments, the memory 904 can include both volatile and nonvolatile memory and data storage components. Volatile components are those that do not retain data values upon loss of power. Nonvolatile components are those that retain data upon a loss of power. Thus, the memory 904 can include, for example, a RAM, ROM, magnetic or other hard disk drive, solid-state, semiconductor, or similar drive, USB flash drive, memory card accessed via a memory card reader, floppy disk accessed via an associated floppy disk drive, optical disc accessed via an optical disc drive, magnetic tape accessed via an appropriate tape drive, and/or other memory component, or any combination thereof. In addition, the RAM can include, for example, a static random access memory (SRAM), dynamic random access memory (DRAM), or magnetic random access memory (MRAM), and/or other similar memory device. The ROM can include, for example, a programmable read-only memory (PROM), erasable programmable read-only memory (EPROM), electrically erasable programmable read-only memory (EEPROM), or other similar memory device.

The processor 902 can be embodied as one or more processors 902 and the memory 904 can be embodied as one or more memories 904 that operate in parallel, respectively, or in combination. Thus, the local interface 906 facilitates communication between any two of the multiple processors 902, between any processor 902 and any of the memories 904, or between any two of the memories 904, etc. The local interface 906 can include additional systems designed to coordinate this communication, including, for example, a load balancer that performs load balancing.

As discussed above, model engine 826 can be embodied, at least in part, by software or executable-code components for execution by general purpose hardware. Alternatively the same can be embodied in dedicated hardware or a combination of software, general, specific, and/or dedicated purpose hardware. If embodied in such hardware, each can be implemented as a circuit or state machine, for example, that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc.

Also, any logic or application described herein, including the model engine 826 that are embodied, at least in part, by software or executable-code components, can be embodied or stored in any tangible or non-transitory computer-readable medium or device for execution by an instruction execution system such as a general purpose processor. In this sense, the logic can be embodied as, for example, software or executable-code components that can be fetched from the computer-readable medium and executed by the instruction execution system.

The computer-readable medium can include any physical media such as, for example, magnetic, optical, or semiconductor media. More specific examples of suitable computer-readable media include, but are not limited to, magnetic tapes, magnetic hard drives, memory cards, solid-state drives, USB flash drives, or optical discs. Also, the computer-readable medium can include a RAM including, for example, an SRAM, DRAM, or MRAM. In addition, the computer-readable medium can include a ROM, a PROM, an EPROM, an EEPROM, or other similar memory device.

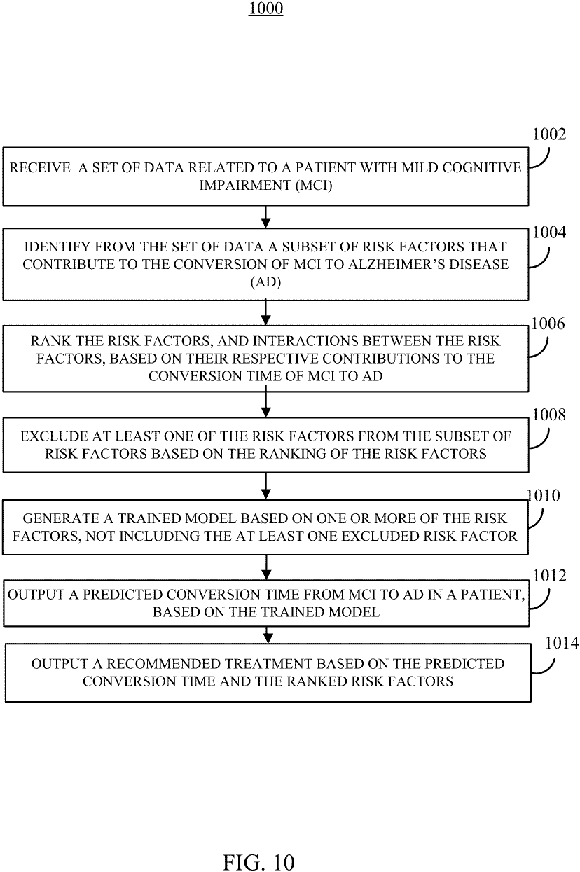

FIG. 10 shows an example 1000 of a process for predicting a conversion time from Mild Cognitive Impairment (MCI) to Alzheimer’s Disease (AD) in patients in accordance with some embodiments of the disclosed subject matter. At 1002, process 1000 can receive a set of data related to a patient with Mild Cognitive Impairment (MCI).

At 1004, process 1000 can identify from the set of data a subset of risk factors (e.g., the risk factors discussed earlier herein with respect to FIG. 2) that contribute to the conversion of MCI to Alzheimer’s disease (AD) in a patient.

At 1006, process 1000 can rank the risk factors, and interactions between the risk factors, based on their respective contributions to the conversion time of MCI to AD. The contributions may be calculated using method disclosed herein.

At 1008, process 1000 can exclude at least one of the risk factors from the subject of risk factors based on the ranking of the risk factors. Further, at least one of the interactions of the risk factors may be excluded based on the ranking of the risk factors. For example, the lowest ranking risk factors and interaction (e.g., the risk factors and interactions with the relatively lowest calculated percentage contributions) may not be necessary for generating a trained model. The inclusion of all risk factors and interactions may overfit a generated model. Therefore, not including all of the data from the data set may be useful for practical applications outside of training and testing a predictive model.

At 1010, process 1000 can generate a trained model based on one or more of the risk factors. The trained model may not include the at least one excluded risk factor. Further, the trained model may not include the at least one excluded interaction between the risk factors. As discussed previously, these exclusions may help to prevent the trained model from being overfit to a set of training data.

The trained model may be a trained machine-learning model where the calculated contributions discussed herein are weighted coefficients determined by one or more neural networks. The one or more neural networks may take as input the risk factors to determine the influence that each risk factor and interaction of risk factors may have on the conversion time of MCI to AD in a patient.

At 1012, process 1000 can output a predicted conversion time from MCI to AD in a patient, based on the trained model. The predicted conversion time may be helpful in recognizing the urgency of a patient’s condition. Further, the predicted conversion time may influence an individual’s quality of life.

At 1014, process 1000 can output a recommended treatment based on the predicted conversion time and the ranked risk factors. Further the recommended treatment may be based on the ranked interactions between the risk factors.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

It should be noted that, as used herein, the term mechanism can encompass hardware, software, firmware, or any suitable combination thereof.

It should be understood that the above-described steps of the processes of FIG. 10 can be executed or performed in any order or sequence not limited to the order and sequence shown and described in the figures. Also, some of the above steps of the processes of FIG. 10 can be executed or performed substantially simultaneously where appropriate or in parallel to reduce latency and processing times.

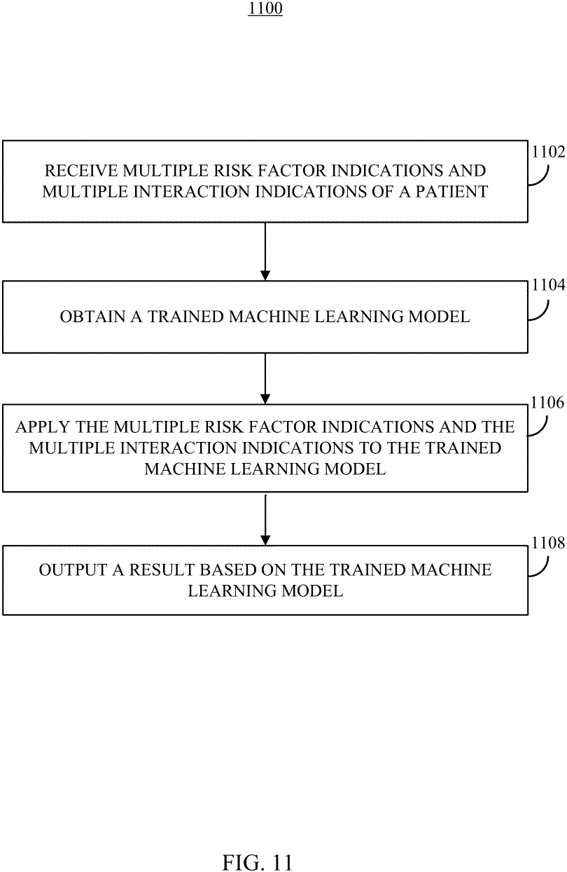

FIG. 11 is a flow diagram illustrating an example process 1100 for disease prediction with some aspects of the present disclosure. As described below, a particular implementation can omit some or all illustrated features/steps, may be implemented in some embodiments in a different order, and may not require some illustrated features to implement all embodiments. In some examples, an apparatus or a computing device 900 with the processor 824 or 902 with memory 820 or 904 in connection with FIGS. 8 or 9 can be used to perform example process 1100. However, it should be appreciated that any suitable apparatus or means for carrying out the operations or features described below may perform process 1100.

At step 1102, process 1100 can receive multiple risk factor indications and multiple interaction indications of a patient. In some examples, each interaction indication of the multiple interaction indications can be an indication of interaction between two risk factor indications of the multiple risk factor indications. In some examples, the multiple risk factor indications can include at least one selected from the group of: an age indication, an education indication, a ventricles indication (or other cardiovascular indication from, e.g., an imaging of a patient’s heart), a hippocampus indication, an entorhinal indication, a fusiform indication, an amyloid-beta indication, a tau indication, a pTau indication, and an Alzheimer Disease Assessment Scale (ADAS) indication for the patient.

In further examples, the multiple risk factor indications can be at least one selected from the group of: clinical data, genetic data, biospecimen data, and medical image data. In some scenarios, the clinical data can include at least one selected from the group of: a cognitive assessment score, an omega-3 score, or demographic statistics. In further scenarios, the genetic data can include: Apolipoprotein E (APOE) genotyping. In even further scenarios, the biospecimen data can include at least one selected from the group of: a quantity of P-tau protein, tau protein, or Beta-amyloid. In further scenarios, the medical image data can be obtained (e.g., from an MRI image, a CT scan image, an x-ray image, an ultrasound image, etc.). The medical image data include at least one selected from the group of: hippocampus, ventricles, entorhinal, intracranial volume (ICV), and fusiform MRI image data. In further examples, the multiple interaction indications include at least one selected from the group of: a first interaction indication between amyloid-beta and tau, a second interaction indication between hippocampus and pTau, a third interaction indication between ventricles and pTau, and a fourth interaction indication between hippocampus and education. In some examples, each risk factor indication can be a numeric value converted from clinical data, genetic data, biospecimen data, or medical image data. In further example, process 1100 can receive each risk factor indication as a numeric data type. Thus, process 1100 can receive clinical data, genetic data, biospecimen data, and medical image data as a numeric data type. In even further examples, an interaction indication between first and second risk factor indications indicates that the effect of one predictor variable (i.e., the first risk factor indication) on the response variable is different at different values of the other predictor variable (i.e., the second risk factor indication). For example, the effect of Hippocampus on time of conversion is different at different values of the pTau and that can be explained be the weighted coefficients (e.g., 0.0045 in the model). In further examples, the multiple risk factor indications and the multiple interaction indications can be data at the time the patient is diagnosed with MCI. However, it should be appreciated that the multiple risk factor indications and the multiple interaction indications are not limited to the time the patients are diagnosed with MCI. The multiple risk factor indications and the multiple interaction indications can be any time between the diagnosis with MCI and the diagnosis with AD.

For example, process 1100 can receive clinical information, generic information, biospecimen, and/or a medical image from a user input or from a third party (e.g., hospital). Then, process 1100 can produce risk factor indications based on the clinical information, generic information, biospecimen, and/or the medical image. Then, process 1100 can calculate interaction indications based on the risk factor indications. Thus, the user input can be the minimum number of individual factors that the user has to input in order to provide the algorithm with the solo risk factor indications and interaction indications necessary.

In further examples, process 1100 performs pre-processing of the multiple risk factor indications and multiple interaction indications. For example, process 1100 can transform a first probability distribution of the multiple risk factor indications and multiple interaction indications to a second probability distribution to follow a normal distribution. For the transformation, process 1100 can use an ordered quantile transformer to transform the probability distribution of the response variable (e.g., the multiple risk factor indications and multiple interaction indications) to the normal or Gaussian probability distribution. For example, if x represents the original data (i.e., original multiple risk factor indications and multiple interaction indications), the transformation formula can be defined as:

$$g \left(x\right) = {φ}^{- 1} \left(\frac{R a n k \left(x\right) - 0.5}{l e n g t h \left(x\right)}\right) ,$$

where φ denotes the standard normal cumulative distribution function, rank(x) is the rank of each observation, and length(x) refers to the number of observations. Here, an observation indicates the values of the response variables (e.g., the multiple risk factor indications and multiple interaction indications).

In some examples, process 1100 can receive clinical information, generic information, biospecimen, and/or a medical image from a user input or from a third party (e.g., hospital). Then, process 1100 can convert the clinical information, the generic information, biospecimen, and/or the medical image to runtime data. In some examples, the runtime data is intermediate data to be transformed to the multiple risk factor indications and multiple interaction indications to follow a normal distribution. Thus, the runtime data can be prior risk factor indications and prior interaction indications to be transformed to follow a normal distribution.

At step 1104, process 1100 can obtain a trained machine learning model. In some examples, the trained machine learning model can include a multivariate linear regression machine learning model. However, it should be appreciated that the machine learning model is not limited to the multivariate linear regression machine learning model. As one example, a machine learning model can be configured as a feedforward network, in which the connections between nodes do not form any loops in the network. As another example, a machine learning algorithm can be configured as a recurrent neural network (“RNN”), in which connections between nodes are configured to allow for previous outputs to be used as inputs while having one or more hidden states, which in some instances may be referred to as a memory of the RNN. RNNs are advantageous for processing time-series or sequential data. Examples of RNNs include long-short term memory (“LSTM”) networks, networks based on or using gated recurrent units (“GRUs”), or the like.

The machine learning model can be structured with different connections between layers. In some instances, the layers are fully connected, in which each all of the inputs in one layer are connected to each of the outputs of the previous layer. Additionally or alternatively, neural networks can be structured with trimmed connectivity between some or all layers, such as by using skip connections, dropouts, or the like. In skip connections, the output from one layer jumps forward two or more layers in addition to, or in lieu of, being input to the next layer in the network. An example class of neural networks that implement skip connections are residual neural networks, such as ResNet. In a dropout layer, nodes are randomly dropped out (e.g., by not passing their output on to the next layer) according to a predetermined dropout rate. In some embodiments, a machine learning algorithm can be configured as a convolutional neural network (“CNN”), in which the network architecture includes one or more convolutional layers. In some embodiments, process 1100 can use tensor flow lite to deploy the machine learning algorithm to a mobile device.

At step 1106, process 1100 can apply the multiple risk factor indications and the multiple interaction indications to the trained machine learning model. In further examples, the multivariate linear regression machine learning model can be defined as: the result = β₀ +∑ᵢ αᵢxᵢ + ∑ⱼγⱼkⱼ + εᵢ, wherein β₀ is an intercept of the multivariate linear regression machine learning model, αᵢ is a first coefficient of i^{th} individual risk factor indication xᵢ of the multiple risk factor indications, γⱼ is a second coefficient of j^{th} interaction indication kⱼ of the multiple interaction indications, and εᵢ is a residual error of the multivariate linear regression machine learning model. In further examples, the first coefficient and the second coefficient were determined during a training phase of the multivariate linear regression machine learning model.

At step 1108, process 1100 can output a result based on the trained machine learning model. In some examples, the result comprises a predicted conversion time of Mild Cognitive Impairment (MCI) to Alzheimer’s Disease (AD) in the patient. In further examples, the result can further include an effectiveness indication of a drug in increasing or decreasing the predicted conversion time in the patient.

In some examples, process 1100 can be used in a medical diagnosis application to provide the predicted conversion time of MCI to AD in a patient. In some examples, the medical diagnosis application can be used by a medical professional in a hospital or a patient at any suitable place. For example, the patient or the medical professional can provide patient medical information (e.g., risk factor indications, interaction indications, clinical data, genetic data, biospecimen data, or MRI images, etc.) to the medical diagnosis application. In other examples, the diagnosis application is connected to, is synchronized with, or electrically coupled with another system or database to access or retrieve the patient information. In some examples, the diagnosis application can provide a predicted conversion time of MCI to AD in the patient, track predicted conversion times in the patient, show an effective indication of a drug for AD treatment, and/or track an effective indications of one or more drugs for AD treatment. In further examples, the medical diagnosis application can display or transmit a notification when the predicted conversion time is shorter than a predetermined period of time, the change of the predicted conversion time is faster than a threshold change rate, etc. In even further examples, the notification can be directly sent to a medical professional associated with the patient for an appointment with the medical professional. In further examples, a user of an app might enter their information and provide outcome data that could be added to the training set.

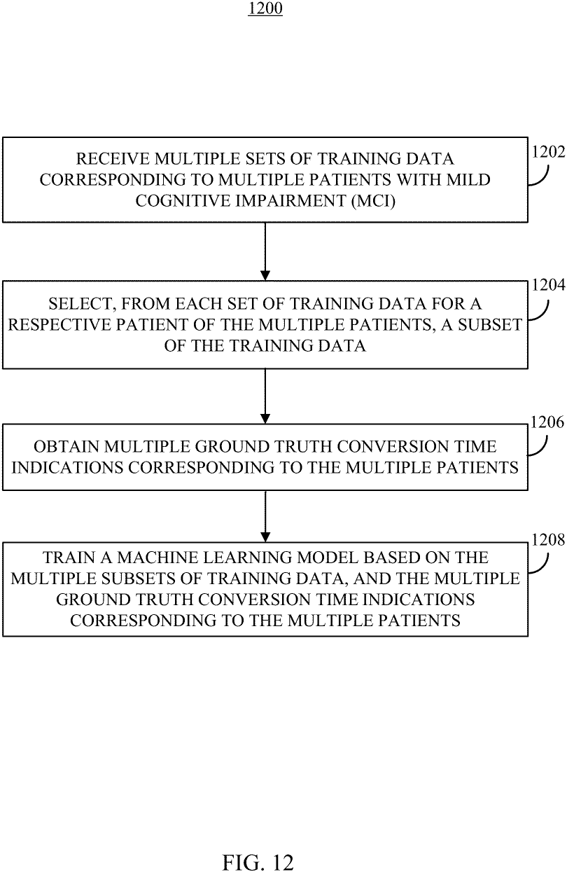

FIG. 12 is a flow diagram illustrating an example process 1200 for disease prediction model training with some aspects of the present disclosure. As described below, a particular implementation can omit some or all illustrated features/steps, may be implemented in some embodiments in a different order, and may not require some illustrated features to implement all embodiments. In some examples, an apparatus or a computing device 900 with the processor 824 or 902 with memory 820 or 904 in connection with FIGS. 8 or 9 can be used to perform example process 1200. However, it should be appreciated that any suitable apparatus or means for carrying out the operations or features described below may perform process 1200.

At step 1202, process 1200 can receive multiple sets of training data corresponding to multiple patients with Mild Cognitive Impairment (MCI). Thus, each set of training data can correspond to a patient. In some examples, a set of training data can include a superset of risk factor indications and a superset of interaction indications. In further examples, the training data can be at the time the patients are diagnosed with MCI. However, it should be appreciated that the training data is not limited to the time the patients are diagnosed with MCI. The training data can be any time between the diagnosis with MCI and the diagnosis with AD.

At step 1204, process 1200 can select, from each set of training data for a respective patient of the multiple patients, a subset of the training data. In some examples, the subset can include: multiple risk factor indications and multiple interaction indications for the respective patient. In further examples, the subset from each set of training data can be multiple subsets of training data. Thus, process 1200 can select for a patient a subset (multiple risk factor indications and multiple interaction indications) of the training data from a corresponding set (i.e., a superset of risk factor indications and a superset of interaction indications) of the training data. In further examples, the rationale to select the subset of training data is based on the amount of contribution to the conversion time from MCI to AD. For example, the amounts (%) of contribution to the conversion time from rank 1 to rank 14 are hippocampus (12.4%), pTau (11.3%), fusiform (10.7%), tau (10%), amyloid-beta (abeta) (9.7%), interaction between abeta and tau (8.8%), ventricles (6.8%), interaction between hippocampus and pTau (6.1%), entorhinal (5.3%), Alzheimer’s Disease Assessment Scale (ADAS13) (4.5%), age (3.7%), interaction between hippocampus and education (3.2%), interaction between ventricles and pTau (1.5%), and education (0.4%). Thus, summing up these amounts of the risk factors and interactions results in 94% contribution to the conversion time. Thus, the quantity of the final analytical model in predicting the conversion time from MCI to AD is 94.4% accurate. However, it should be appreciated that more risk factors and/or interactions can be added to the selected training data and/or some risk factors and/or interactions can be removed from the selected training data.

At step 1206, process 1200 can obtain multiple ground truth conversion time indications corresponding to the multiple patients. For example, the process 1200 can monitor the multiple patients for a period (e.g., 8 years, any suitable years, or until each patient is diagnosed with AD) and record the conversion times for the patients. The conversion times for the patients are ground truth data to be used in a machine learning model at step 1208.

At step 1208, process 1200 can train a machine learning model based on the multiple subsets of training data, and the multiple ground truth conversion time indications corresponding to the multiple patients. In some examples, the machine learning model can include a multivariate linear regression machine learning model. The multivariate linear regression machine learning model can be defined as: a predicted conversion time indication for the respective patient = β₀ + Σᵢαᵢxᵢ + Σⱼγⱼkⱼ + εᵢ. Here, β₀ is an intercept of the multivariate linear regression machine learning model, αᵢ is a first coefficient of i^{th} individual risk factor indication xᵢ of the multiple risk factor indications, γⱼ is a second coefficient of j^{th} interaction indication kⱼ of the multiple interaction indications, and εᵢ is a residual error of the multivariate linear regression machine learning model. In further examples, to train the machine learning model, process 1200 can adjust a coefficient of the machine learning model based on a cost function and the multiple ground truth conversion time indications. Here, the coefficient to be adjusted can include the first coefficient and the second coefficient. In some examples, process 1200 can adjust the coefficient using a cost function (e.g., root means square errors (RMSE)), which is given by:

$$R M S E = \sqrt{\frac{{\sum }_{i = 1}^{n} {\left({y}_{i} - {\hat{y}}_{i}\right)}^{2}}{n}} ,$$

where yᵢ is a ground truth conversion time indication of patient index i, ŷᵢ is a predicted conversion time of patient index i, and n is the total sample size (total number of patients). Thus, the machine learning model can be trained by comparing the ground truth conversion times for corresponding patients and estimated conversion times, which are outputs from the machine learning model, and adjusting the coefficient values based on the comparison.

In further examples, process 1200 transform a first probability distribution of multiple inputs or outputs of the machine learning model to a second probability distribution. For the transformation, process 1200 can use an ordered quantile transformer to transform the probability distribution of the response variable (e.g., training data, or inputs/outputs of the machine learning model) to the normal or Gaussian probability distribution. For example, if x represents the original data (i.e., outputs of the machine learning model), the transformation formula can be defined as:

$$g \left(x\right) = {φ}^{- 1} \left(\frac{R a n k \left(x\right) - 0.5}{l e n g t h \left(x\right)}\right) ,$$

where φ denotes the standard normal cumulative distribution function, rank(x) is the rank of each observation, and length(x) refers to the number of observations. Here, an observation indicates the values of the response variables (e.g., training data or inputs/outputs of the machine learning model). In some examples, the ordered quantile transformer can be performed as a pre-processing step of training data before training the machine learning model such that the response variables (e.g., training data) to follow a normal distribution. Thus, the ordered quantile transformation can improve the modeling performance and the relationship with the input risk factors.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A system for cognitive disease prediction, comprising:
a memory; and
a processor communicatively coupled to the memory;
wherein the memory stores a set of instructions which, when executed by the processor, cause the processor to:
receive a plurality of risk factor indications for a given patient and determine a plurality of interaction indications of the patient, each interaction indication of the plurality of interaction indications being an indication of interaction between at least two risk factor indications of the plurality of risk factor indications;
obtain a trained machine learning model;
apply the plurality of risk factor indications and the plurality of interaction indications to the trained machine learning model; and
output a prediction of conversion to the cognitive disease for the patient based on the trained machine learning model.

2. The system of claim 1, wherein the plurality of risk factor indications includes at least one selected from a group of: clinical data, genetic data, biospecimen data, and medical image data.

3. The system of claim 2, wherein the clinical data includes at least one selected from a group of: a cognitive assessment score, an omega-3 score, or demographic statistics.

4. The system of claim 2, wherein the genetic data includes: Apolipoprotein E (APOE) genotyping.

5. The system of claim 2, wherein the biospecimen data includes at least one selected from a group of: a quantity of P-tau protein, tau protein, or Beta-amyloid.

6. The system of claim 2, wherein the medical image data includes at least one selected from a group of: hippocampus, ventricles, entorhinal, intracranial volume (ICV), and fusiform medical image data.

7. The system of claim 1, wherein the plurality of risk factor indications comprises at least one selected from a group of: an age indication, an education indication, a ventricles indication, a hippocampus indication, an entorhinal indication, a fusiform indication, an amyloid-beta indication, a tau indication, a pTau indication, and an Alzheimer Disease Assessment Scale (ADAS) indication for the patient.

8. The system of claim 1, wherein the plurality of interaction indications comprises at least one selected from a group of: a first interaction indication between amyloid-beta and tau, a second interaction indication between hippocampus and pTau, a third interaction indication between ventricles and pTau, and a fourth interaction indication between hippocampus and education.

9. The system of claim 1, wherein the trained machine learning model comprises a multivariate linear regression machine learning model.

10. The system of claim 9, wherein the multivariate linear regression machine learning model is defined as:
the result = β₀ +∑ᵢ αᵢxᵢ + ∑ⱼ γⱼkⱼ + εᵢ,
wherein β₀ is an intercept of the multivariate linear regression machine learning model, αᵢ is a first coefficient of i^{th} individual risk factor indication xᵢ of the plurality of risk factor indications, γⱼ is a second coefficient of j^{th} interaction indication kⱼ of the plurality of interaction indications, and εᵢ is a residual error of the multivariate linear regression machine learning model.

11. The system of claim 10, wherein the first coefficient and the second coefficient were determined during a training phase of the multivariate linear regression machine learning model.

12. The system of claim 1, wherein the result comprises a predicted conversion time of Mild Cognitive Impairment (MCI) to Alzheimer’s Disease (AD) in the patient.

13. The system of claim 12, wherein the result further comprises an effectiveness indication of a drug in increasing or decreasing the predicted conversion time in the patient.

14. A system for disease prediction model training, comprising:
a memory; and
a processor communicatively coupled to the memory;
wherein the memory stores a set of instructions which, when executed by the processor, cause the processor to:
receive a plurality of sets of training data corresponding to a plurality of patients with Mild Cognitive Impairment (MCI);
select, from each set of training data for a respective patient of the plurality of patients, a subset of the training data, the subset comprising: a plurality of risk factor indications and a plurality of interaction indications for the respective patient, the subset from each set of training data being a plurality of subsets of training data;
obtain a plurality of ground truth conversion time indications corresponding to the plurality of patients; and
train a machine learning model based on the plurality of subsets of training data, and the plurality of ground truth conversion time indications corresponding to the plurality of patients.

15. The system of claim 14, wherein to select, from each set of training data for the respective patient, the subset of the training data, the set of instructions causes the processor to:
rank contribution data in each set of training data based on contribution of the contribution data to a respective conversion time indication of the respective patient; and
select a subset of the contribution data for the respective patient in each set of training data for the respective patient based on the ranked contribution data, the subset of the contribution data being the subset of the training data.

16. The system of claim 14, wherein to train the machine learning model, the set of instructions causes the processor to:
adjust a coefficient of the machine learning model based on a cost function and the plurality of ground truth conversion time indications.

17. The system of claim 14, wherein to train the machine learning model, the set of instructions further causes the processor to: transform a first probability distribution of pre-training data to the plurality of sets of training data to follow a second probability distribution, and wherein the second probability distribution is a normal probability distribution.

18. The system of claim 14, wherein the plurality of risk factor indications comprises at least one selected from a group of: an age indication, an education indication, a ventricles indication, a hippocampus indication, an entorhinal indication, a fusiform indication, an amyloid-beta indication, a tau indication, a pTau indication, and an Alzheimer Disease Assessment Scale (ADAS) indication for the respective patient, and
wherein the plurality of interaction indications comprises at least one selected from a group of: a first interaction indication between amyloid-beta and tau, a second interaction indication between hippocampus and pTau, a third interaction indication between ventricles and pTau, and a fourth interaction indication between hippocampus and education for the respective patient.

19. The system of claim 14, wherein the machine learning model comprises a multivariate linear regression machine learning model.

20. The system of claim 19, wherein the multivariate linear regression machine learning model is defined as:
a predicted conversion time indication for the respective patient = β₀ +∑ᵢ αᵢxᵢ + ∑ⱼγⱼkⱼ + εᵢ,
wherein β₀ is an intercept of the multivariate linear regression machine learning model, αᵢ is a first coefficient of i^{th} individual risk factor indication xᵢ of the plurality of risk factor indications, γⱼ is a second coefficient of j^{th} interaction indication kⱼ of the plurality of interaction indications, and εᵢ is a residual error of the multivariate linear regression machine learning model.

21. A system for predicting conversion of mild cognitive impairment to Alzheimer’s disease in a patient, comprising:
a memory; and
a processor communicatively coupled to the memory;
wherein the memory stores a set of software instructions which, when executed by the processor, cause the processor to:
receive, from a user, clinical data, genetic data, biospecimen data, and medical image data of the patient;
transform the clinical data, genetic data, biospecimen data, and medical image data to runtime data;
transform the runtime data to a plurality of risk factor indications, to follow a normal distribution;
determine a plurality of interaction indications of a patient, each interaction indication of the plurality of interaction indications being an indication of interaction between at least two risk factor indications of the plurality of risk factor indications;
obtain a trained machine learning model;
apply the plurality of risk factor indications and the plurality of interaction indications to the trained machine learning model;
output a predicted time to conversion from mild cognitive impairment to Alzheimer’s disease for the patient based on the trained machine learning model; and
track the result based on the trained machine learning model for a predetermined period of time.
